(19) United States
(12) Patent Application Publication
(10) Pub. No.: US 2015/0038694 A1
(43) Pub. Date: Feb. 5, 2015

(54) GLUCOSE DERIVATIVES BOUND TO ARSENIC FOR USE IN THE TREATMENT OF TUMOUR

(71) Applicants: Marco SALVETTI, Roma (IT); Francesco NICOTRA, (US); Giovanni RISTORI, Roma (IT); Barbara LA FERLA, Milano (IT); Giovanni CODACCI PISANELLI, Roma (IT)

(72) Inventors: Francesco Nicotra, Milano (IT); Barbara La Ferla, Milano (IT); Marco Salvetti, Roma (IT); Giovanni Codacci Pisanelli, Roma (IT); Giovanni Ristori, Roma (IT); Michele Pitaro, Rome (IT)

(73) Assignee: XENUS SRL, Roma (IT)

(21) Appl. No.: 14/374,294

(22) PCT Filed: Feb. 19, 2013
(86) PCT No.: PCT/IT2013/000051

§ 371 Date: Jul. 24, 2014

(30) Foreign Application Priority Data
Feb. 20, 2012 (IT) RM2012A000058

(51) Int. Cl. A61K 47/48; A61K 31/555

(52) U.S. Cl. CPC A61K 47/48092; A61K 31/555
USPC 536/54

(57) ABSTRACT

A family of sugar-based molecules for therapeutic use, includes compounds with a structure having at least one sugar molecule with carrier function, bound to arsenic having active principle function, allowing the selective therapeutic treatment of tumoral pathologies and not, distinguished by a high cellular metabolism and, consequently, by a high glucose consumption. Based on the preferential glucose absorption according to the Warburg effect, the compounds are adapted to penetrate inside tumoral cells and not, distinguished by a high cellular metabolism, via the glucose molecule integrated in their structure, so as to determine the selective therapeutic treatment of the cells and, consequently, of the pathologies deriving therefrom, by the arsenic molecule chemically bound to above mentioned glucose molecule. Processes for the production of synthetic sugar-based compounds and extracted sugar-based compounds, produced from natural sources are also described.

The present invention concerns the field of drugs and medical devices per the selective therapeutic treatment of tumoral pathologies and not.

More in detail, the present invention concerns a family of sugar-based molecules for the selective therapeutic treatment of tumoral pathologies and not, wherein said pathologies are distinguished by a high cellular metabolism and, consequently, a high consumption of glucose.

As it is known since 1926 from the studies of the German biochemist Otto Warburg, the malignant tumor cells have a glucose catabolism that differs from the one of healthy cells in that their cellular metabolism is based on the preferential use of glucose in glycolysis, and in the following minimum exploitation of the tricarboxylic acids cycle, also in the presence of oxygen (Warburg effect).

The preferential use of glucose by tumoral cells is triggered by oncogenes and tumor suppressor genes, it starts early in the carcinogenesis and seems to increase with tumor aggressiveness.

Even if said process is functional to the expansion of the tumor in the body, by the production of intermediate molecules useful to the growth and division of the tumoral cells, it is absolutely detrimental from the energetic point of view, because only two of the thirty-six ATP-molecules (Adenosine triphosphate) produced by a glucose molecule are obtained by glycolysis.

Due to said energetic inefficiency, the tumoral cells are forced to import high quantities of glucose through the cytoplasmic membrane, consequently increasing the expression of specific integral membrane glycoproteins (GLUT—glucose transporters), suitable for transferring inside said tumoral cells the glucose necessary for their sustenance.

The same phenomenon was observed in presence of non tumoral pathologies, like e.g. some viral infections.

In the cells infected by cytomegalovirus, e.g., a diversion of the citrate occurs from the Krebs cycle towards the synthesis of fatty acids, with the same increase of glucose demand that occurs in the oncogenesis (Yu et al. Trends Microbiol. 2011).

Furthermore, in the context of the preferential use of glucose in cases of infection, recently it has been observed that “persister” bacteria, i.e. bacteria resistant to antibiotic treatments, which are an important public health problem, can be eradicated inducing therein a greater use of glucose, or of other intermediate metabolites, given together with said antibiotic (Allison et al. Nature 2011).

The recent use of 2-(¹⁸F)-fluorine-2-deoxy-D-glucose (FDG), as a tracer in the positron emission tomography (PET), confirms in vivo the accumulation of glucose in the tumoral cells and is the main medical application of the Warburg effect.

Above mentioned tracer, mainly consisting of glucose molecules chemically associated to radioactive fluorine molecules, is preferentially absorbed by the tumoral cells, through the glucose cells present in the structure thereof, and allows the detection of the accumulation of said glucose in the concerned tumoral cells, through the radioactive fluorine molecules associated to said substance, thus favouring the acquisition of clinical data important for diagnostics and stadiation of tumors in the body.

In a way similar to above described diagnostic use, the preferential inlet of glucose in tumoral cells can be obviously exploited also for therapeutic purposes.

The efforts of chemically binding glucose to common antitumor drugs, like e.g. cyclophosphamide, however have generated compounds which, due to the steric bulk of said drugs, do not penetrate the cell through the GLUT but through the co-transporter sodium-glucose (SAAT1), thus determining a lower therapeutic selectivity of said compounds towards tumoral cells, deriving from the impossibility of advantageously exploiting above mentioned GLUT overexpression.

Even the use of glucose radioactive compounds, like anti-neoplastic agents, did not lead to favourable clinical results due to the suboptimal efficiency and the difficulty of administration of these substances.

It is the aim of the present invention to realize a family of sugar-based molecules suitable for transporting a therapeutic agent, of the non radioactive type, inside tumoral cells and not, distinguished by a high cellular metabolism, for allowing the selective therapeutic treatment of said cells and, consequently, of the pathologies deriving therefrom, advantageously exploiting the principle of the preferential absorption of the sugars, and in particular of glucose, according to above mentioned Warburg effect.

The aim set forth is reached by the design and synthesis of compounds in which a sugar molecule, in particular a glucose molecule, is bound to arsenic in different oxidation states, through a covalent binding, directly or through spacers, and exploiting the different sugar hydroxyl positions, including the anomeric one, or also replacing the same.

Therefore, a first object of the present invention is a family of sugar-based molecules for therapeutic use according to the main independent claim 1.

A preferred embodiment of the present invention concerns a compound for therapeutic use, having the following general formula of structure (I):

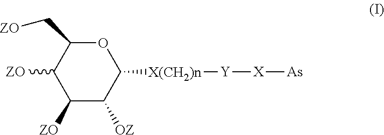

wherein:
X is a CH₂ group or an aromatic ring Ar, or an O atom or an S atom;
n varies from 1 to 10;
Y is a NH group, or a NHSO₂ group, or a NHSO group, or a NHCO group, or a S atom, or an O atom or a CH═CH group;
As is any derivate arsenic, in any oxidation condition like e.g.:

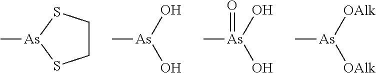

Z is an H atom, or X(CH₂)ₙ—Y—X—As as previously described.

A second object of the present invention is a process for the production of sugar-based molecules for therapeutic use, by means of a synthesis process.

A third object of the present invention is a process for the production of sugar-based molecules for therapeutic use by means of a process of extraction from natural sources.

Further features and advantages of the present invention will be described hereinbelow in the following specification.

Relating to the general formula (I), the family of sugar-based molecules according to the present invention mainly consists of a sugar molecule, in particular a glucose molecule, chemically bound, through a covalent binding, to any arsenic derivative, in any oxidation state.

The covalent binding between said glucose molecule and said arsenic molecule is obtained directly or through spacers, and exploiting the different hydroxyl positions of sugar, including the anomeric one, or even replacing the same.

The structure of the sugar-based molecules according to the present invention has been confirmed by mass spectrometry (MS) and nuclear magnetic resonance (NMR).

Above mentioned family of sugar-based molecules allows the therapeutic treatment of tumoral pathologies and not, distinguished by a high cellular metabolism, exploiting the principle of the preferential absorption of glucose according to the Warburg effect.

In fact, said molecules are suitable for preferentially penetrating inside tumoral cells and not, distinguished by a high cellular metabolism, by means of the glucose molecule integrated in their structure, thus determining the selective therapeutic treatment of said cells and consequently of the pathologies deriving therefrom, by means of the arsenic molecule chemically bound to above mentioned glucose molecule.

The penetration of the glucose, and of the arsenic chemically bound thereto, inside tumoral cells and not, distinguished by a high cell metabolism, is advantageously favoured by GLUT overexpression present on the cytoplasmic membrane of the same.

The preferential absorption of glucose by above mentioned cells determines the consequent inlet in the same of a proportional quantity of arsenic, such as to induce ad advantageous therapeutic effect in said cells whilst being comparatively harmless for healthy cells.

Furthermore, in the presence of tumoral cells the amount of arsenic absorbed by those cells will be such as to determine the selective suppression thereof, while in presence of cells suffering from pathologies of inflammatory kind the amount of arsenic absorbed by said cells will be such as to determine the sole therapeutic treatment.

The use of arsenic as therapeutic agent solves the problem of the detection of a substance that:
is pharmacologically active;
has a steric bulk such as not to alter its passage through overexpressed GLUT on the cytoplasmic membrane of tumoral cells and not, distinguished by a high cellular metabolism;
is able to interfere with the metabolism of said cells due to its chemical similarity with phosphorus.

In fact, arsenic has an appropriate steric bulk, it is commonly used in form of trioxide in the treatment of human malignant tumors like promyelokytic leukemia, and its anti-inflammatory and chemiotherapeutic activity is well known in medicine due to its use in the therapeutic treatment of syphilis and many other pathologies. Furthermore, arsenic, always in form of trioxide, is able to increase radiosensitivity of solid tumors.

### EXAMPLE 1

### Production of Sugar-Based Compounds by a Synthesis Process

By way of example, a compound according to the present invention is shown, identified by GD152.186 with the following formula of structure (S):

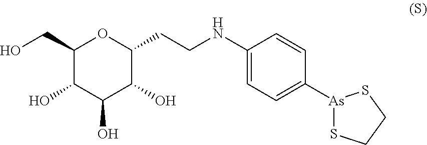

The compound GD152.186 has been produced by means of a non-limitative synthesis process, shown in the following scheme:

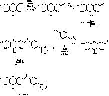

Said synthesis process comprises the following steps:
a preparation step of a solution of allil-C-acetylated glucose, obtained by stereoselective allylation performed according to the procedure described by Gray (Bennek, 1987);
an acetylation step of above mentioned derivative by means of acetylation in pyridine;
an ozonolysis reaction performed by dissolution in CH₂Cl₂, cooling of the solution to −78° C. and the saturation thereof with ozone;
a resting phase of said solution, of seventy-five minutes;
a saturation step of said solution, performed with oxygen first and then with nitrogen;
a step of triphenylphosphine addition to said solution, followed by rising to room temperature and following shaking, suitable for helping the conversion to aldehyde;
an aldehyde reduction ammination step, performed with any derivative of arsenic, in any oxidation condition, and with NaCNBH₃ (for obtaining above mentioned compound GD152.186, derivative of arsenic b has been used);
a purification step of the compound thus obtained;
a step of deacetylation of said compound, performed by means of MeONa in MeOH dry, arranged for providing said product in its final form;
a characterization phase: ¹H NMR (400 MHz, CD₃OD) δppm 7.29-7.21; (m, 1H), 7.04-6.94; (m, 1H), 6.60-6.46; (m, 2H), 4.00-3.90; (m, 1H), 3.75; (bd, J =11.78; Hz, 1H), 3.56-3.47; (m, 2H), 3.44-3.35; (m, 2H), 3.23-3.06; (m, 7H), 1.90-1.81; (m, 2H). ¹³C NMR (100 MHz, CD₃OD) 166.7, 153.0, 135.8, 132.8, 117.0, 116.2, 78.79, 78.03, 77.60, 75.67, 75.27, 66.08, 44.94, 44.80, 27.77; MS calcd for C₁₆H₂₄AsNO₅S₂ [M+H]⁺ 450: found 450.

### EXAMPLE 2

### Antitumor Activity of the Sugar-Based Molecules Family According to the Present Invention

The compound GD152.186 (PM 449,4) produced by means of above mentioned synthesis process, has been tested in a series of preclinical testing on cellular lines of human tumors, and in particular on a cellular line of human neuroblastoma (SK-N-BE) and on a cellular line of human promyelokytic leukemia (HL60).

The culture medium used for growing the SK-N-BE cells is DMEM High Glucose (Dulbecco's Modified Eagle Medium) with 10% FBS (fetal bovine serum), 1% L-Glutamine and 1% Penicillin/Streptomycin antibiotics.

Instead, the HL60 cells grow in suspension at a temperature of 37° C. and in 5% CO₂, and the culture medium used is RPMI 1640 with 10% FBS (fetal bovine serum), 1% L-Glutamine and 1% Penicillin/Streptomycin antibiotics.

As a positive control, arsenic trioxide (As₂O₃, Sigma-Aldrich) has been used, as its activity has already been tested on cellular lines of human neuroblastoma and human promyelokytic leukemia.

The compound GD152.186 has been dissolved in sterile water so as to reach the final concentrations of 100-300-500-1000-2000 μM.

The test used for evaluating the persistence of tumor cells after the contact with said compound GD152.186 is the MTT test (reduction of tetrazolium salts).

Said test is based on the ability of the mitochondrial respiratory enzyme of the cells, usually called succinate tetrazolium reductase, of reducing a tetrazolium salt (3-[4,5-dimethylthiazol-2-yl]-2,5-diphenyltetrazolium bromide (MTT, Sigma Aldrich)) into an insoluble compound of blue colour, usually called formazan.

Said reduction process occurs only in living cells and not in dead or damaged ones, consequently, the amount of formazan produced is directly proportional to the number of cells metabolically active in the tested sample.

96-well plates have been used for performing the tests, wherein in each well 5.000 (SK-N-BE) cells or 50.000 (HL60) cells have been sown, suspended in 180 μL of medium and then incubated at 37° C. and 5% CO2.

Forty-eight hours later, the cells have been treated with 20 μL of growing concentrations (in six replicates) of arsenic trioxide (100-300-500-1000 μM) and of GD152.186 compound (100-300-500-1000-2000 μM), so as to obtain inside each single well the final concentrations of 10-30-50-100 and 200 μM.

To one six-well column no compound is added but only 20 μL of sterile water, for following the natural growth of the cells.

The plates are incubated again for forty-eight, seventy-two and ninety-six hours, at the end thereof the test is performed.

To each single well 20 μL of MTT solution (5 mg·m-1 in sterile FBS) have been added, giving an incubation time of three hours, functional to the formation of formazan.

Then, the supernatant is removed from each well and 200 μL of DMSO are added.

After about ten minutes the concentration level of formazan has been taken by means of a spectrophotometer set at a wave length of 570 nm.

In those cells treated with arsenic trioxide, morphologic changes have been found, visible with an optical microscope, like the loss of adherence to the plate and the presence of a roundish shape with irregular outlines.

The formazan absorbance data have shown that the reduction of the cell survival is strictly dependent on the arsenic trioxide dosage and on the time of exposure to said substance.

Also in the cells treated with GD152.186 compound morphologic changes have been surprisingly detected, like the loss of adherence to the plate and the presence of roundish shape with irregular outlines.

Said changes have been detected above all in presence of high concentrations of GD152.186 compound.

Surprisingly, the formazan absorbance data have confirmed that the reduction of the cell survival is directly depending on the dosage of the GD152.186 compound and on the time of exposure to said substance.

The reduction of the cell survival according to the time of exposure to GD152.186 compound (respectively of forty-eight, seventy-two and ninety-six hours) has been most visible when the cells have come into contact with high concentrations (100-200 μM) of said substance.

Furthermore, the formazan absorbance data concerning the HL60 cells have shown a reduction of the cell survival when the same have been put into contact with increasing concentrations of arsenic trioxide and of GD152.186 compound.

The clinical data obtained through above described testing form a fundamental check that if arsenic is chemically bound to glucose compounds having affinity with GLUTs, it maintains a strongly cytotoxic activity towards tumoral cells.

The preferential absorption of glucose by tumoral cells, according to the principle defined by the Warburg effect, also reveals a greater therapeutic importance of sugar-based compounds according to the present invention, compared to arsenic trioxide and similar substances, commonly used in anti-tumoral treatments.

On the base of the clinical data obtained by above mentioned testing, the sugar-based compounds according to the present invention are suitable for finding an advantageous application in the selective therapeutic treatment of tumoral pathologies, distinguished by a high cellular metabolism, such as neoplasias derived from epithelia (e.g. carcinomas, adenocarcinomas, etc.), of mesenchymal origin (e.g. fibrosarcomas, liposarcomas, rhabdomyosarcomas, osteosarcomas, etc.), of the blood cells (e.g. leukaemias, lymphomas, myelomas, etc.) or of the nervous tissue (e.g. astrocytomas, glioblastomas, meningiomas, gangliocytomas, etc.).

### EXAMPLE 3

Production of Sugar-Based Compounds by Means of a Process of Extraction from Natural Sources

Beyond being obtained by means of synthesis processes, the compounds according to the present invention may also be extracted from organisms such as algae, molluscs, fungi, bacteria, rice and more.

Natural sugas-based compounds may also be advantageously used for the therapeutic treatment of tumoral pathologies and not, distinguished by a high cellular metabolism.

Being the capacity of ribose of penetrating the citoplasmic membrane of tumoral cells and not, through the GLUTs, widely known, it appears logic to assume that natural sugar-based compounds also have an adequate antimetabolic activity.

In fact, most sugar-based natural compounds [J. Feldmann, E. M. Krupp, Anal. Bioanal. Chem. 2011, 399, 1735-1741] identified so far, have a pentavalent arsenic directly bound to the carbonaceous backbone of sugar, to two alkyl substituents (usually methyls) and to one atom of oxygen and/or sulphur (thioarsenosugars). Such compounds may differ in the substituent bound in anomeric position, usually a unit of glycerol, differently functionalized in the primary hydroxyl with different substituents (sulphates, phosphates, sulfonates).

For the extraction of sugar-based compounds from natural sources procedures may be used commonly employed for the extraction of sugars from organic matrixes, properly adapted and optimized according to the features of the specific organic matrix treated.

By way of a non-limiting example, a process of extraction of sugar-based compounds from natural sources comprises:
a step of shredding of the chosen organic matrix, repeatedly treated with water so as to save the water-soluble components, among which the compounds of interest;
a step of concentration of the obtained solution, performed by evaporation at reduced pressure, possibly associated to lyophilisation;
a step of separation and purification of the solutes, containing the compounds of interest, from the liquid component of the solution mentioned above, performed by means of chromatographic and/or crystallization techniques.

### EXAMPLE 4

### Anti-Inflammatory Activity of the Family of Sugar-Based Molecules According to the Present Invention

On the base of the preferential absorption principle of glucose according to the Warburg effect, the sugar-based compounds obtained by synthesis and the extracted compounds according to the present invention are suitable for having an advantageous application also in the therapeutic treatment of inflammatory pathologies of infective kind, distinguished by a high cellular metabolism, such as viral infections due to increase glucose consumption (Yu et al. Trend Microbiol. 2011).

In particular, said compounds are adapted to selectively hit cells infected by viruses that have an increased uptake of glucose with respect to healthy cells, deriving from the replacement of the glucose carrier commonly used by the same, as in the case of cells infected by cytomegalovirus, wherein GLUT4 replaces GLUT1 determining an increase of sugars flow and of the density of the carrier on the cell surface (Yu et al. Trend Microbiol. 2011).

Furthermore, said compounds are adapted to have advantageous application in the context of preferential use of glucose in case of infection by “persister” bacteria.

In fact, it is known that said bacteria resistant to antibiotic treatments, are usually treated inducing therein a greater use of glucose, or of other intermediate metabolites thereof, given together with said antibiotic so as to favour the absorption thereof (Allison et al. Natura 2011).

1) A family of sugar-based molecules for therapeutic use, characterized in that it comprises compounds with a structure having at least one sugar molecule, having carrier function, bound to arsenic having active principle function.

2) A compound for therapeutic use according to claim 1, characterized in that it has the following general formula of structure (I): 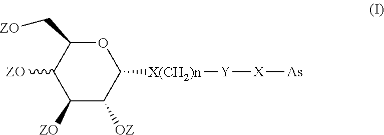
wherein:
X is a CH₂ group or an aromatic ring Ar, or an O atom, or an S atom;
n varies from 1 to 10;
Y is a NH group, or a NHSO₂ group, or a NHSO group, or a NHCO group, or an S atom, or an O atom or a CH═CH group;
As is any derivative of arsenic, in any oxidation condition like e.g.: 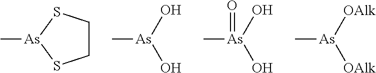
Z is an H atom, or X(CH₂)ₙ—Y—X—As as previously described.

3) A compound according to claim 2, characterized in that the arsenic derivative is chemically bound to a glucose molecule by means of a covalent bond.

4) A compound according to claim 3, characterized in that said covalent bond is realized directly or by means of spacers, and exploiting the different hydroxylic positions of sugar, comprising the anomeric one, or even replacing the same.

5) A compound according to claim 1, characterized in it is adapted to allow the selective therapeutic treatment of tumoral pathologies, distinguished by a high cellular methabolism, such as neoplasias derived from epithelia (e.g., carcinomas, adenocarcinomas, etc.), of mesenchymal origin (e.g. fibrosarcomas, liposarcomas, rhabdomyosarcomas, osteosarcomas, etc.), of the blood cells (e.g. leukaemias, lymphomas, myelomas, etc.). or of the nervous tissue (e.g. astrocytomas, glioblastomas, meningiomas, gangliocytomas, etc.).

6) A compound according to claim 1, characterized in that it is adapted to allow the selective therapeutic treatment of non tumoral pathologies, distinguished by a high cellular metabolism, like viral infections due to increased glucose consumption (e.g., cytomegalovirus infections, persister bacteria infections, etc.).

7) A process for the production of sugar-based synthetic compounds for therapeutic use, characterized in that it comprises:
a preparation step of a solution of allil-C-acetylated glucose, obtained by stereoselective allylation performed according to the procedure described by Gray (Bennek, 1987);
an acetylation step of above mentioned derivative by means of acetylation in pyridine;
an ozonolysis reaction performed by dissolution in CH₂CH₂, cooling of the solution to −78° C. and the saturation thereof with ozone;
a resting phase of said solution, of seventy-five minutes;
a saturation step of said solution, performed with oxygen first and then with nitrogen;
a step of triphenylphosphine addition to said solution, followed by rising to room temperature and following shaking, suitable for helping the conversion to aldehyde;
an aldehyde reduction ammination step, performed with any derivative of arsenic, in any oxidation condition, and with NaCNBH₃;
a purification step of the compound thus obtained;
a step of deacetylation of said compound, performed by means of MeONa in MeOH dry, arranged for providing said product in its final form;
a characterization phase: ¹H NMR (400 MHz, CD₃OD) δ ppm 7.29-7.21; (m, 1H), 7.04-6.94; (m, 1H), 6.60-6.46; (m, 2H), 4.00-3.90; (m, 1H), 3.75; (bd, J=11.78 Hz, 1H), 3.56-3.47; (m, 2H), 3.44-3.35; (m, 2H), 3.23-3.06; (m, 7H), 1.90-1.81; (m, 2H). ¹³C NMR (100 MHz, CD₃OD) 166.7, 153.0, 135.8, 132.8, 117.0, 116.2, 78.79, 78.03, 77.60, 75.67, 75.27, 66.08, 44.94, 44.80, 27.77; MS calcd for C₁₆H₂₄AsNO₅S₂ [M+H]⁺ 450: found 450.

8) A process for the production of sugar-based extractive compounds from natural sources for therapeutic use, characterized in that it comprises:
a selection step of the preferred organic matrix;
a step of shredding of the chosen organic matrix, repeatedly treated with water so as to save the water-soluble components, among which the compounds of interest;
a step of concentration of the obtained solution, performed by evaporation at reduced pressure, possibly associated to lyophilization;
a step of separation and purification of the solutes, containing the compounds of interest, from the liquid component of the solution mentioned above, performed by means of chromatographic and/or crystallization techniques.

9) A process according to claim 8, characterized in that the preferred organic matrix is chosen among algae, molluscs, fish, fungi, bacteria, rice.
